(19) United States
(12) Patent Application Publication
(10) Pub. No.: US 2008/0249327 A1
(43) Pub. Date: Oct. 9, 2008

(54) Catalyst for the Synthesis of Dimethyl Carbonate in the Gas Phase

(76) Inventors: Reinhard Eckelt, Berlin (DE); Martin J.G. Fait, Berlin (DE); Rolf Fricke, Berlin (DE); Manfred Richter, Konigs Wusterhausen (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/814,543

(22) PCT Filed: Jan. 23, 2006
(86) PCT No.: PCT/EP2006/050363

§ 371 Date: Mar. 12, 2008

(30) Foreign Application Priority Data
Jan. 24, 2005 (DE) 10 2005 003 879.4

(51) Int. Cl. C07C 68/00; C01B 39/02

(52) U.S. Cl. 558/277; 423/700

(57) ABSTRACT

The invention relates to an improved catalyst for the synthesis of dimethyl carbonate by reacting methanol, carbon monoxide and oxygen in the gas phase and to the use thereof. The catalyst consists of a copper-containing zeolite produced by admixing one or more halide-free copper(II) compounds to a zeolite in a liquid medium, drying the zeolite modified by the admixture, and tempering at 400-900° C. under inert conditions, essentially retaining the crystallinity of the zeolite, said admixing being effected by means of a method selected from the group consisting of impregnation of the zeolite, ion exchange, precipitation of copper(II) hydroxide in the presence of the zeolite, and a combination of these methods. The catalyst shows high space-time yields, is constant over the period of operation and has no corrosive action.

### FIELD OF THE INVENTION

The invention relates to an improved catalyst for the synthesis of dimethyl carbonate by reacting methanol, carbon monoxide and oxygen in the gas phase and to the use thereof.

### BACKGROUND OF THE INVENTION

The suitability of dimethyl carbonate (DMC) as environmentally friendly solvent and methylating agent in chemical processes, as fuel additive replacing methyl t-butyl ether (MTBE), and as a base chemical in polycarbonate production has led to an increasing demand on the world market. Substantial growth rates can be expected from the use of polycarbonates and the development on the market of optical memories.

In technical terms, the oxidative carbonylation of methanol to form DMC has been accomplished in the form of the gas-liquid slurry process by EniChem (CuCl/activated carbon as catalyst) and in the form of the UBE process (2-stage process via methyl nitrite on PdFeCuCl/activated carbon).

2CH₃OH+2NO+½O₂→2CH₃ONO+H₂O (1)

2CH₃ONO+CO→CH₃OCOOCH₃+2NO (2)

In addition to the use of CO, this route involves operations with nitrogen oxides.

The element copper as an active catalyst component dominates the patent literature relating to gas-liquid phase processes. Cu(I) and/or Cu(II) can be used in oxidative carbonylation of methanol in liquid phase. According to the current state of knowledge, the presence of copper halides is important because other copper salts are inactive. This also applies to those cases where nitrogen-containing ligands or polyamide support materials simultaneously acting as ligands for Cu(II) are used.

For oxidative carbonylation of methanol in the gas phase, catalysts containing copper are suitable as well. Cu(II) is predominantly used in the form of CuCl₂ in order to modify a support, mostly activated carbon, by means of an impregnation process.

U.S. Pat. No. 5,907,051 describes catalysts based on activated carbon as support material impregnated with copper salt solutions. The materials impregnated with CuCl show the best space-time yields (STY) of DMC (about 70 g/(l_{catalyst}·h). Exemplary catalysts produced using copper acetate, copper oxalate and copper borate solutions have a maximum STY of DMC of 40 g/(l_{catalyst}·h). EP 607,943 reports an STY of DMC of 67 g/(l_{catalyst}·h). The test period in all of the above cases was only 4 hours, so that the stability of the catalyst system cannot be assessed. In addition, one essential drawback of activated carbon supports is the limited temperature window available for secondary treatment. Calcination in a stream of air without oxidation or inflammation is only possible up to a maximum of 400° C. However, this temperature is not sufficient to produce an active copper phase from the copper compounds following impregnation, unless a halide, e.g. in the form of chloride, is also present. For this reason, the best STYs of DMC are ultimately obtained on activated carbons impregnated with copper chloride, although the problems of halide-containing catalyst systems remain unresolved.

Processes using salt melts as catalysts, preferably including copper(I) chloride and potassium chloride, likewise represent highly aggressive and corrosive catalyst systems.

Another drawback of catalysts containing metal chlorides is emission of hydrogen chloride, thereby causing a reduction in the service life of the catalysts, as well as corrosion of the plant and pollution of the environment. While addition of chlorohydrocarbons or hydrogen chloride to the reactants can reduce loss of activity, such process managing entails additional problems of corrosion in the plants.

For continuous oxidative carbonylation of methanol in the gas phase, U.S. Pat. No. 5,391,803 suggests Cu-containing zeolites produced by heating a solid copper compound in the presence of a zeolite. Production then proceeds by dry grinding or sublimation of the Cu(I) compound. In the examples (molar composition of the reactants methanol/CO/O₂/N₂=0.88/4/0.5/2), a methanol conversion of 3-5% at 130° C. is achieved at normal pressure and a gas volume load of 870 h⁻¹, with a maximum selectivity of 80% for the target product, resulting in a space-time yield (STY) of DMC of about 140 g/(l_{catalyst}·h). Here, CuCl is mostly used as solid in the preparation, so that a halide-containing catalyst variant is used in this case as well. When using Cu₂O as solid, a methanol conversion of only 2% is achieved, so that this catalyst appears to have little activity.

As is apparent from the prior art, it is important to do without halide-containing additives both in production of the catalysts and in conducting the reaction and, at the same time, achieve stable activity and high selectivity for the DMC target product. The crucial importance of univalent copper ions to the oxidative carbonylation of methanol can be regarded as a secure fact.

Direct modification of a suitable zeolite by ion exchange with Cu(I) is difficult due to the low solubility of Cu(I) salts in aqueous solutions and their tendency to disproportionate into Cu(0) and Cu(II). Likewise, solid exchange using halide-free Cu(I) compounds fails to provide catalysts having sufficient activity and selectivity.

The basic object of the invention is therefore to develop an active and selective catalyst maintaining said properties over a long period of time and, at the same time, avoid corrosion in the apparatus system by halides.

### SUMMARY OF THE INVENTION

Surprisingly, it was found that Cu(II)-containing zeolites can be modified with advantage by using a specific activation procedure, allowing the use thereof as active and selective catalysts in the oxidative carbonylation of methanol into DMC in the gaseous phase even at normal pressure.

In this process, a precursor produced via halide-free Cu(II) salts in a liquid medium by impregnation of the zeolite, ion exchange, precipitation of copper hydroxide/oxide in the presence of zeolite or combinations of the above three variants is subsequently treated using inert tempering, water vapor in an inert medium or combinations of these variants at sufficiently high temperatures under normal pressure or in vacuum.

According to the invention, the catalyst is a crystalline or partially amorphous aluminum silicate having the composition (based on the anhydrous form):

[Cu(I)ₐ,H_{b},Me(I)_{c},Me(II)_{d},Me(III)ₑ][(AlO₂)_{f}(SiO₂)_{g}] (I),

wherein 1≦a≦f, 0≦b≦f−1, Me(I) represents a univalent cation such as Ag, Li, Na, K, Rb, Cs with 0≦c≦f−1, Me(II) represents a divalent cation such as Zn, Co, Fe, Be, Mg, Ca, Sr, Ba or Ni with 0≦d≦(f−1)/2, and Me(III) represents a trivalent cation such as Co, Fe, Cr, La with 0≦e≦(f−1)/3, the sum of the indices is a, b, c, d, e=f, the index f may assume values of from 4 to 58, and the silicon-to-aluminum ratio g/f varies from 1 to 100, and wherein Cu(I) represents a summary mean oxidation number of copper, independently of the actual oxidation numbers Cu(0), Cu(I) or Cu(II), and the content of copper in the zeolite ranges from 1 to 20 wt.-%, obtainable by impregnation or ion exchange of a zeolite in a liquid aqueous phase with halide-free copper(II) compounds, or precipitation of Cu(II) hydroxide from an aqueous phase in the presence of said zeolite, or impregnation, ion exchange and precipitation, calcination of the produced Cu-containing zeolite in air at 300-500° C., and activation of the Cu-containing zeolite to give the summary mean oxidation number Cu(I) of copper in formula (I) by treatment with an inert gas at 600-900° C. or with a mixture of inert gas and water vapor at 300-900° C. until a white or virtually white solid is obtained.

Temperatures of 600-900° C. for inert tempering and temperatures of 400-700° C. for water vapor treatment were found advantageous.

No halide is required for the catalyst produced according to the invention, neither in production nor in maintaining catalytic activity and selectivity.

Using the halide-free catalysts according to the invention, it is possible to accomplish the oxidative carbonylation of methanol in the gas phase in a temperature range of 120-220° C. under normal pressure, at gas volume loads of 500-5000 l/(l_{catalyst}·h) and with STYs of DMC of 50-250 g/(l_{catalyst}·h).

The reaction under elevated pressure on the catalyst results in a further increase of the DMC yield and likewise represents a variant of the invention.

### DETAILED DESCRIPTION OF THE INVENTION

The inventive catalyst for the synthesis of dimethyl carbonate in the gas phase is characterized by a copper-containing zeolite produced by admixing one or more halide-free copper(II) compounds to a zeolite in a liquid medium, drying the zeolite modified by the admixture, and tempering at 400-900° C. under inert conditions, essentially retaining the crystallinity of the zeolite, said admixing being effected by means of a method selected from the group consisting of impregnation of the zeolite, ion exchange, precipitation of copper(II) hydroxide in the presence of the zeolite, and a combination of these methods.

The liquid medium is preferably water or a mixture of water and water-miscible organic compounds, such as alcohols, ketones, ethers and polyethylene glycol derivatives.

The halide-free copper(II) compounds are advantageously selected from the group consisting of copper(II) complexes, e.g. copper(II) tetrammine complexes; salts of inorganic acids, such as copper(II) nitrate, copper(II) sulfate, copper(II) hydrogen carbonate; as well as salts of organic acids and hydroxycarboxylic acids, e.g. copper(II) formate, copper(II) acetate, copper(II) tartrate, copper(II) lactate, each of which being soluble in the liquid medium.

“Impregnation” of the zeolite is understood to be complete immersion of the solid zeolite particles in appropriate impregnating solutions, as well as wetting the solid particles with the impregnating solution (incipient wetness method).

Tempering is preferably effected under inert conditions in the presence of nitrogen or noble gas at 400-900° C. for a time period of from 0.1 to 100 h, and tempering can also be effected in the presence of water vapor at any concentration from 0.1 to 99%. Preferred water vapor contents are around 5 to 80 vol.-%. Preferred tempering ranges are around 600-900° C., especially 600-700° C. in the presence of inert gas and water vapor, or 710-850° C. in the presence of inert gas only.

Inert tempering can also be achieved in a microwave field or in vacuum.

Following production of the modified zeolite (precursor), the latter is dried and calcined in air at about 300-500° C. The crucial step in obtaining the catalyst according to the invention is removal of acidic centers (Brønsted-acidic centers) remaining in the precursor after modification with copper. These acidic centers stabilize Cu(II) ions and prevent the necessary change in valency of copper during the catalytic reaction. The consequence of this—as has been found—would be low activity of such catalysts.

On the other hand, the use of non-Brønsted-acidic solids for the production of the precursor is not successful. Modification of the zeolites by copper requires acidic centers allowing migration of Cu(II) ions and fixing of copper ions in the zeolite lattice. Therefore, it is only after the modification, i.e., starting from the precursor, that the centers can be removed, and migration of copper ions may also proceed in parallel with the activation procedure.

This surprising fact has led to the inventive method of removing Brønsted-acidic centers following ion exchange which requires the initial presence of such centers. Removal of the Brønsted-acidic centers is effected by thermal treatment in a flow of inert gas at a temperature resulting in dehydroxylation of the Brønsted-acidic centers, thereby destabilizing Cu(II). This process is crucial to facilitate the required redox processes with formation of the catalytically active univalent state of copper under reaction conditions.

In another embodiment of the invention the protons of the Brønsted-acidic centers remaining after ion exchange can be replaced with alkali and/or alkaline earth cations in a further ion exchange.

One precondition for the inventive activation and the resulting active catalyst is to prepare a suitable precursor. Although the above-mentioned methods of modifying zeolites with copper represent the state of knowledge, operations resulting in suitable precursors for the activation according to the invention will be described below.

For ion exchange, the zeolite can be used in the alkali form (i.e., negatively charged centers of the crystalline aluminum silicate lattice are compensated by alkali ions such as Na⁺, K⁺, also in mixture with others), ammonium form (where charge compensation is effected by NH₄⁺ ions) or proton form (H⁺ represents the charge-compensating counterion).

In the alkali form of the zeolite, ion exchange can be effected using aqueous solutions of Cu(II) complexes. Addition of excess ammonia results in the formation of Cu(II) tetrammine cations, so that precipitation of Cu(II) hydroxide is largely avoided. Depending on the concentration conditions, the exchange process produces a mixed alkali CuNH₄ form which is converted into the alkali CuH form upon calcination in air.

Also, other suitable complexing agents (e.g. ethanolamine, methylamine) can be used instead of ammonia.

Exchange in the NH₄⁺ form of the zeolite can be effected using aqueous solutions of Cu(II) complexes (Cu(II) tetrammine). Ammonia is added in excess, so that precipitation of Cu(II) hydroxide is largely avoided. Following completion of the exchange process, a modified form of the zeolite is produced which, depending on the degree of exchange, includes ammonium ions and copper ions. During calcination in air, the ammonium ions undergo decomposition with elimination of ammonia, and the zeolite is converted into the CuH form. The maximum achievable degree of exchange is limited to about 50%.

Exchange in the H form of the zeolite can be effected using aqueous solutions of Cu(II) salts (e.g. Cu(II) nitrate, acetate, sulfate). Addition of ammonia or other bases to raise the pH value to 5-6 gives rise to partial precipitation of Cu(II) hydroxide which can be reacted in a normal calcination (air, 400-500° C.) with H⁺ to form dispersed Cu(II) cations. This process proceeds with exchange levels of up to 100% (Cu/Al≦0.5). At exchange levels of more than 100%, part of the copper is invariably present as CuO.

During ion exchange, part of the cations (H⁺, NH₄⁺, alkali) bound to Brønsted-acidic centers in the zeolite will be replaced by Cu(II) cations. In the event of complete exchange, the required charge compensation allows a molar Cu(II)/Al ratio of 0.5, corresponding to an exchange level of 100% of the cations (H⁺, NH₄⁺, alkali) bound to Brønsted-acidic centers in the zeolite. If the desired degree of exchange is selected to be higher than 100%, some CuO will be present following preparation and oxidative calcination.

All of the precursors produced according to one of the methods described have only little catalytic activity in an oxidative carbonylation of methanol to form DMC, as will be demonstrated in Reference Example 1 below.

In a first embodiment the precursor is activated by inert tempering, optionally preceded by additional steps, such as reduction, among other things, which reduction is preferably effected using hydrogen. The conditions (temperature, time and hydrogen concentration) are selected in such a way that a mean oxidation state of Cu of about +1 is achieved. That is, when accounting for the amounts of all Cu species, a summary mean Cu oxidation number of about +1 should be achieved. In this context, it is not a crucial matter how much Cu is present as Cu(0), Cu(I) or Cu(II) and which types of species (e.g. Cu—O—Cu , CuO) are present The crucial point is that a mean oxidation state of copper of close to +1 can develop as a result of comproportionation and autoreduction reactions proceeding during subsequent inert tempering (up to the limit of thermal stability of the zeolite). This is supported by the pre-reduction. Simultaneously, excess Brønsted centers are converted into Lewis centers by thermal dealumination, so that the Cu(II) cations are no longer stabilized. Pre-reduction and inert tempering result in formation of a white solid wherein a very high amount of copper is present as Cu(I) cation.

In formula (I) illustrated below, “Cu(I)” therefore represents a mean oxidation state of copper of close to +1 which also involves other oxidation states of copper.

The high redox activity of the catalyst according to the invention can be seen in color changes from white to blue/green caused by oxidation and hydration of Cu(I) into Cu(II), which appear within a few seconds at room temperature upon exposure to air.

Another embodiment starts from a precursor produced by precipitation of copper hydroxide on NH₄—Y zeolite at a mass ratio of copper to aluminum of from 0.9 to 1.1. Activation of this precursor is effected by means of a water vapor treatment at 650° C. for a time of 5 h using a stream of inert gas (volume flow rate: 100 cm³/min) having a water vapor content of 37%.

In a preferred fashion, a zeolite having a faujasite type structure (Linde Y) with an idealized composition of the (anhydrous) Na form of Na₅₈[Al₅₈Si₁₃₄O₃₈₄] is suitable for the preparation of the precursor. The molar Si/Al ratio for this composition is 2.3, the Na content is calculated to be 10.4%. The concentration of Brønsted-acidic centers is 5.03 mmol of H₊/g (anhydrous H form); the Na form contains 4.53 mmol of Na⁺ (anhydrous).

Following activation according to the invention, the catalyst generally consists of a modified crystalline or partially amorphous zeolite having the composition (based on the anhydrous form):

[Cu(I)ₐ,H_{b},Me(I)_{c},Me(II)_{d},Me(III)ₑ][(AlO₂)_{f}(SiO₂)_{g}] (I),

wherein 1≦a≦f, 0≦b≦f−1, Me(I) represents a univalent cation such as Ag, Li, Na, K, Rb, Cs with 0≦c≦f−1, Me(II) represents a divalent cation such as Zn, Co, Fe, Be, Mg, Ca, Sr, Ba or Ni with 0≦d≦(f−1)/2, and Me(II) represents a trivalent cation such as Co, Fe, Cr, La with 0≦e≦(f−1)/3, the sum of the indices a, b, c, d, e=f, the index f may assume values of from 4 to 58, and the silicon-to-aluminum ratio g/f varies from 1 to 100, and wherein Cu(I) represents a summary mean oxidation number of copper, independently of the actual oxidation numbers Cu(0), Cu(I) or Cu(II). However, due to the varying oxidation states of copper that are present, the representation of the catalyst by formula is merely a conditional characterization.

If the zeolite structure includes further metal ions, these are preferably Ag, Na or K for Me(I), Mg, Ca, Ni or Co for Me(II), and Fe for Me(III). Different metal ions of same or different valency may also be included.

The silicon-to-aluminum ratio g/f is preferably 2-25, especially 2-6.

In general, wide-pore zeolites with entry openings of 12 Al—Si tetrahedrons are suitable as zeolites, e.g. those from the groups of BEA, e.g. Beta; MOR, e.g. mordenite; FAU, e.g. X or Y; MAZ, e.g. mazzite or Omega; LTL, e.g. zeolite L as well as medium-pore zeolites with entry openings of 10 Al—Si tetrahedrons, e.g. those from the group of MFI, e.g. ZSM-5.

In a preferred fashion, a suitable precursor is produced by means of ion exchange starting from dilute Cu(II) salt solutions, among which copper(II) acetate is preferred, and a faujasite zeolite structure, preferably zeolite Na—Y with an idealized composition of Na₅₈[Al₅₈Si₁₃₄O₃₈₄]. The copper content of the samples ranges from 1 to 20%, preferably from 5 to 15%.

Another feature of the catalysts according to the invention, when modified by copper only and in the absence of further cations changing the appearance in color, such as Mn, Co, Fe, Ni and Cr, is that the inactive form (precursor) has a distinct color, mostly blue, green, blue-gray, gray-green or blue-green, whereas the activated form is white to virtually white.

Another advantageous embodiment of the invention is that the activated catalyst is mixed with an inert material of good heat conductivity of at least 10 W/m·Kelvin in order to improve heat distribution. In a preferred fashion the activated catalyst is ground with the inert filler at a ratio of from 1:10 to 1:0.5, especially from 1:3 to 1:1, and subsequently employed in the catalytic reaction in powdered form or in any shape. The inert filler is preferably graphite. Other materials having good heat conductivity and being essentially inert at the reaction temperatures of the subsequent catalytic reaction can also be used.

The invention also relates to the use of a copper-containing zeolite catalyst produced by admixing one or more halide-free copper(II) compounds to a zeolite in a liquid medium, drying the zeolite modified by the admixture, and tempering at 400-900° C. under inert conditions, essentially retaining the crystallinity of the zeolite, said admixing being effected by means of a method selected from the group consisting of impregnation of the zeolite, ion exchange, precipitation of copper(II) hydroxide in the presence of the zeolite, and a combination of these methods, in a catalytic process for the synthesis of dimethyl carbonate by direct carbonylation of methanol with carbon monoxide and oxygen, wherein the reaction of methanol with carbon monoxide and oxygen to form dimethyl carbonate is effected at temperatures of from 120 to 220° C., advantageously from 130 to 170° C., pressures of 1-25 bars, and a gas volume load of from 500 to 5,000 h⁻¹, preferably from 1,000 to 3,000 h⁻¹.

The oxidative carbonylation of methanol in the gas phase can be performed at an overall pressure of 1 bar. However, it is convenient to operate at elevated pressure, e.g. 2-60 bars, preferably 5-25 bars, in order to have a sufficiently high reaction rate.

In general, the reaction temperature is about 110° C. to 300° C., preferably 120 to 220° C., and typical reaction temperatures are around 130-170° C.

In general, the composition of the reaction mixture of methanol, carbon monoxide and oxygen can be varied within wide limits, observing the explosion limits when metering the amounts. Operations can optionally be performed in the presence of inert gases. The data given in the examples were obtained with a molar composition of MeOH/CO/O₂/Ar/He=0.36/0.48/0.06/0.05/0.05.

It should be noted that the molar ratios of the reaction components employed are important to the reaction rate and the selectivity of the reaction, the predominant byproducts being dimethyl ether and dimethoxymethane.

Depending on the pressure and temperature, the gas load can be varied within wide limits, but is preferably 500 to 5000 h⁻¹, so that STYs of 50-250 g of DMC/(l_{catalyst}·h) can be achieved.

To have markedly higher space-time yields of up to 250 g of DMC/(l_{catalyst}·h) at 1 bar overall pressure, constant STY over the time of operation, avoiding corrosive influences by halide-free operation, means a significant improvement of the catalyst of the invention in the process of producing dimethyl carbonate.

The invention will be illustrated in the following examples and with reference to a drawing.

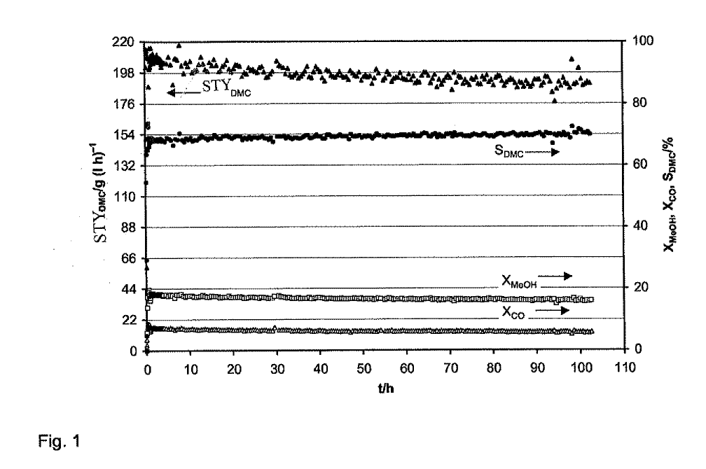

FIG. 1 shows the catalytic results of oxidative carbonylation of methanol into DMC in the gas phase at 1 bar overall pressure for an operation period of 100 h on the catalyst formulation of Example 5.

### EXAMPLE 1

Preparation of the modified zeolite (precursor): A solution of 2.0 g of Cu(II) acetate in 100 g of distilled water in a sealable vessel at room temperature is added with 5 ml of ammonia solution (25%) with stirring with a magnetic stirrer. Owing to the formation of the Cu(II) tetrammine complex, the color of the solution turns from light-blue into a deep dark-blue. The pH value should be about 9. Following addition of 5.0 g of Na—Y zeolite (dried at 120° C.), the vessel is sealed, and this is stirred for 24 h at 25° C. Thereafter, suction filtration is effected, and the blue precipitation is washed three times with 50 ml of ammonia solution (0.5%). The Cu(II)NH₄Na—Y zeolite is initially dried at room temperature and subsequently at 120° C. Finally, this is calcined in air at 400° C. The Cu(II)HNa form being formed is gray-green at 400° C. and green-blue in cooled and rehydrated state (humidity). The Cu content of the catalyst referred to as precursor is 7.8% as determined by chemical analysis.

Activation: To produce the catalyst described for use in the process according to the invention, 2 g of the blue-green granulate is heated in a stream of inert gas (50 ml/min, Ar with O₂<10 ppm) from room temperature to 800° C. at a heating rate of 10 K/min, held at this temperature for 15 h, and subsequently cooled down.

The catalyst A thus obtained is exposed to a mixture of MeOH/CO/O₂/Ar/He=0.36/0.48/0.06/0.05/0.05 (GHSV: 3000h⁻¹) in a tubular flow reactor under normal pressure. Depending on the temperature, the values given in Table 1 are obtained.

TABLE 1
Oxidative carbonylation of methanol into DMC on catalyst A of Example 1

| | | | | | | STY_{DMC} |
|---|---|---|---|---|---|---|
| T (° C.) | X_{MeOH} (%) | X_{CO} (%) | S_{DMC} (%) | S_{DMM} (%) | S_{DME} (%) | (g l_{cat}⁻¹ h⁻¹) |
| 130 | 5 | 1 | 72 | 27 | 1 | 68 |
| 140 | 8 | 2 | 69 | 29 | 2 | 99 |
| 150 | 10 | 3 | 66 | 31 | 3 | 126 |
| 160 | 12 | 4 | 63 | 33 | 4 | 142 |
| 170 | 14 | 5 | 51 | 36 | 12 | 130 |

X_{MeOH}: conversion of methanol,
X_{CO}: conversion of CO,
S_{DMC}: selectivity of dimethyl carbonate formation,
S_{DMM}: selectivity of dimethoxymethane formation,
S_{DME}: selectivity of dimethyl ether formation (each based on methanol conversion), and
STY_{DMC}: space-time yield of DMC.

### REFERENCE EXAMPLE 1

Production of the precursor is effected in accordance with Example 1. In contrast to Example 1, the precursor—without further pretreatment—is exposed to a mixture of MeOH/CO/O₂/Ar/He=0.36/0.48/0.06/0.05/0.05 (GHSV: 3000 h⁻¹) in a tubular flow reactor under normal pressure. This precursor B contains 8.2% copper and shows only low activity with respect to the target reaction. The values illustrated in Table 2 are obtained, demonstrating that significant space-time yields cannot be achieved with the modified zeolite without subsequent activation by thermal treatment in the ranges as indicated.

TABLE 2
Oxidative carbonylation of methanol into DMC
on precursor B of Reference Example 1

| | | | | | | STY_{DMC} |
|---|---|---|---|---|---|---|
| T (° C.) | X_{MeOH} (%) | X_{CO} (%) | S_{DMC} (%) | S_{DMM} (%) | S_{DME} (%) | (g l_{cat}⁻¹ h⁻¹) |
| 130 | ≈0 | ≈0 | | | | ≈0 |
| 140 | ≈0 | ≈0 | | | | ≈0 |
| 150 | 4 | 1 | 25 | 17 | 58 | 18 |
| 160 | 7 | 1 | 18 | 16 | 67 | 23 |
| 170 | 10 | 1 | 10 | 15 | 75 | 21 |

X_{MeOH}: conversion of methanol,
X_{CO}: conversion of CO,
S_{DMC}: selectivity of dimethyl carbonate formation,
S_{DMM}: selectivity of dimethoxymethane formation,
S_{DME}: selectivity of dimethyl ether formation (each based on methanol conversion), and
STY_{DMC}: space-time yield of DMC.

### EXAMPLE 2

Example 2 differs from Example 1 in that the precursor is activated using the following procedure:

2 g of the blue-green granulate is treated with 50 ml/min of a reduction gas mixture (argon with 5 vol.-% hydrogen). The temperature is raised from 25 to 350° C. with 10 K/min, and the reducing medium is maintained for 30 min. After switching to inert gas (50 ml/min, Ar with O₂<10 ppm), the temperature is raised to 800° C. at a heating rate of 10 K/min and maintained for 15 h. Thereafter, cooling in a stream of inert gas is effected.

The catalyst C thus obtained has a copper content of 8.3% and is white. Upon exposure to air (oxygen, moisture), the color changes from white to blue-green at room temperature.

This catalyst C is exposed to a mixture of MeOH/CO/O₂/Ar/He=0.36/0.48/0.06/0.05/0.05 (GHSV: 3000 h⁻¹) in a tubular flow reactor under normal pressure. The activity of the catalyst is comparable to that described in Example 1. Depending on the temperature, the values given in Table 3 are obtained.

TABLE 3
Oxidative carbonylation of methanol into DMC on catalyst C of Example 2

| | | | | | | STY_{DMC} |
|---|---|---|---|---|---|---|
| T (° C.) | X_{MeOH} (%) | X_{CO} (%) | S_{DMC} (%) | S_{DMM} (%) | S_{DME} (%) | (g l_{cat}⁻¹ h⁻¹) |
| 130 | 4 | 1 | 72 | 27 | 1 | 55 |
| 140 | 6 | 2 | 69 | 29 | 2 | 82 |
| 150 | 9 | 3 | 65 | 31 | 3 | 105 |
| 160 | 11 | 3 | 59 | 34 | 7 | 117 |
| 170 | 12 | 4 | 50 | 38 | 12 | 110 |

X_{MeOH}: conversion of methanol,
X_{CO}: conversion of CO,
S_{DMC}: selectivity of dimethyl carbonate formation,
S_{DMM}: selectivity of dimethoxymethane formation,
S_{DME}: selectivity of dimethyl ether formation (each based on methanol conversion), and
STY_{DMC}: space-time yield of DMC.

### EXAMPLE 3

Example 3 differs from Example 2 in that the precursor is produced using the proton form of zeolite Y and modified by a second ion exchange with a further element belonging to Group IB of the Periodic Table of the Elements.

A solution of 2.0 g of Cu(II) acetate in 100 g of distilled water in a sealable vessel at room temperature is added with 5 ml of ammonia solution (25%) with stirring with a magnetic stirrer. Owing to the formation of the Cu(II) tetrammine complex, the color of the solution turns from light-blue to deep dark-blue. The pH value should be about 9. Following addition of 5.0 g of H—Y (produced by tempering the ammonium form in a stream of air), the vessel is sealed, and this is stirred for 24 h at 25° C. Thereafter, suction filtration is effected, and the blue precipitation is washed three times with 50 ml of ammonia solution (0.5%).

The CuNH₄ zeolite is initially dried at room temperature and subsequently at 120° C. Finally, this is calcined in air at 400° C. The Cu(II)H form being formed is gray-green at 400° C. and green-blue in cooled and rehydrated state (humidity). The Cu content of the precursor is 6.8% as determined by chemical analysis. The Ag exchange comprises treatment of the precursor (2.0 g) with a solution of 500 mg of AgNO₃ in 50 ml of water. This solution is added with 2 ml of 25% ammonia solution to form the silver diammine complex. The precursor is digested in this solution for 20 h at room temperature. Following suction filtration, washing with distilled water and drying at 120° C., final calcination is effected at 400° C. for 2 h. The precursor thus obtained is heated to 800° C. in a stream of inert gas (50 ml/min, Ar with O₂<10 ppm) at 10 K/min, held at this temperature for 3 h, and subsequently cooled down in the stream of inert gas.

The catalyst D obtained in this way has a copper content of 6.8% and a silver content of 6.4% and is white. Upon exposure to air (oxygen, moisture), the color changes from white to blue-green at room temperature.

This catalyst D is exposed to a mixture of MeOH/CO/O₂/Ar/He=0.36/0.48/0.06/0.05/0.05 (GHSV: 3000 h⁻¹) in a tubular flow reactor under normal pressure. Depending on the temperature, the values given in Table 4 are obtained.

TABLE 4
Oxidative carbonylation of methanol into DMC on catalyst D of Example 3

| | | | | | | STY_{DMC} |
|---|---|---|---|---|---|---|
| T (° C.) | X_{MeOH} (%) | X_{CO} (%) | S_{DMC} (%) | S_{DMM} (%) | S_{DME} (%) | (g l_{cat}⁻¹ h⁻¹) |
| 130 | 6 | 2 | 75 | 24 | 1 | 80 |
| 140 | 8 | 3 | 72 | 27 | 1 | 112 |
| 150 | 11 | 3 | 68 | 29 | 3 | 140 |
| 160 | 12 | 4 | 64 | 31 | 5 | 149 |
| 170 | 13 | 5 | 56 | 33 | 11 | 140 |

X_{MeOH}: conversion of methanol,
X_{CO}: conversion of CO,
S_{DMC}: selectivity of dimethyl carbonate formation,
S_{DMM}: selectivity of dimethoxymethane formation,
S_{DME}: selectivity of dimethyl ether formation (each based on methanol conversion), and
STY_{DMC}: space-time yield of DMC.

### EXAMPLE 4

Example 4 differs from Example 2 in that the ammonium form of zeolite Y is used to produce the precursor. A solution of 2.0 g of Cu(II) acetate in 100 g of distilled water in a sealable vessel at room temperature is added with 5 ml of ammonia solution (25%) with stirring with a magnetic stirrer. Owing to the formation of the Cu(II) tetrammine complex, the color of the solution turns from light-blue to deep dark-blue. The pH value should be about 9. Following addition of 5.0 g of NH₄—Y (dried at 120° C.), the vessel is sealed, and this is stirred for 24 h at 25° C. Thereafter, suction filtration is effected, and the blue precipitation is washed three times with 50 ml of ammonia solution (0.5%). The CuNH₄ zeolite is initially dried at room temperature and subsequently at 120° C. Finally, this is calcined in air at 380° C. for 5 h. The CuH form being formed is gray-green at 400° C. and green-blue in cooled and rehydrated state (humidity).

An amount of 2.0 g of granulated zeolite is heated in a reduction gas mixture (argon with 5 vol.-% hydrogen) from 25 to 350° C. with 10 K/min and held at this temperature for 30 min. After switching to inert gas (50 ml/min, Ar with O₂<10 ppm), the temperature is raised to 800° C. with 10 K/min, held at this temperature for 15 h, followed by final cooling in the stream of inert gas.

The catalyst E has a copper content of 8.2% and is white. The higher copper content results from the special conditions of ion exchange which, when using the ammonium form of the zeolite with identical amounts of available copper, results in higher levels of exchange. Upon exposure to air (oxygen, moisture), the color changes from white to blue-green at room temperature.

The catalyst E is exposed to a mixture of MeOH/CO/O₂/Ar/He=0.36/0.48/0.06/0.05/0.05 (GHSV: 3000 h⁻¹) in a tubular flow reactor under normal pressure. Depending on the temperature, the values given in Table 5 are obtained.

TABLE 5
Oxidative carbonylation of methanol into DMC on catalyst E of Example 4

| | | | | | | STY_{DMC} |
|---|---|---|---|---|---|---|
| T (° C.) | X_{MeOH} (%) | X_{CO} (%) | S_{DMC} (%) | S_{DMM} (%) | S_{DME} (%) | (g/l_{cat} h) |
| 130 | 3 | 1 | 67 | 29 | 4 | 38 |
| 140 | 5 | 1 | 65 | 30 | 6 | 57 |
| 150 | 6 | 2 | 61 | 30 | 9 | 74 |
| 160 | 8 | 2 | 52 | 32 | 16 | 82 |
| 170 | 10 | 3 | 38 | 34 | 29 | 70 |

X_{MeOH}: conversion of methanol,
X_{CO}: conversion of CO,
S_{DMC}: selectivity of dimethyl carbonate formation,
S_{DMM}: selectivity of dimethoxymethane formation,
S_{DME}: selectivity of dimethyl ether formation (each based on methanol conversion), and
STY_{DMC}: space-time yield of DMC.

### EXAMPLE 5

Example 5 differs from Example 4 in that precipitation of copper hydroxide/oxide on NH₄—Y zeolite with a mass ratio of copper to aluminum of about 1 is used to produce the precursor, which precipitation is achieved by increasing the pH value of the suspension by adding tetramethylammonium hydroxide solution (10%) at room temperature.

6.0 g of the NH₄—Y zeolite is suspended in a solution of 1.9 g of Cu(II) acetate in 100 ml of distilled water at room temperature. The light-blue-colored dispersion is added with 19 g of a tetramethylammonium hydroxide solution (10%) at room temperature with intense stirring. The pH value of the suspension rises to 8-9, and the color deepens to dark-blue. Next, this is heated to 80° C., so that the precipitated Cu(OH)₂ is converted to CuO. This process is accompanied by a color change from blue via green to brown. The suction-filtrated solution is colorless and free of Cu (test with NH₃ solution).

The brown precipitation is sucked off and washed three times with 50 ml of distilled water. After drying, calcination is performed (380° C., 1-24 h).

Activation of this precursor is effected by means of a water vapor treatment at 650° C. for a time of 5 h. A stream of inert gas (volume flow rate: 100 cm³/min) having a water vapor content of 37% is passed over 2 g of precursor in a tubular flow reactor.

Following cooling, the catalyst F thus obtained, having a copper content of 12.0%, is exposed to a mixture of MeOH/CO/O₂/Ar/He=0.36/0.48/0.06/0.05/0.05 (GHSV: 3000 h⁻¹) in a tubular flow reactor under normal pressure. Depending on the temperature, the values given in Table 6 are obtained.

TABLE 6
Oxidative carbonylation of methanol into DMC on catalyst F of Example 5

| | | | | | | STY_{DMC} |
|---|---|---|---|---|---|---|
| T (° C.) | X_{MeOH} (%) | X_{CO} (%) | S_{DMC} (%) | S_{DMM} (%) | S_{DME} (%) | (g/l_{cat} h) |
| 130 | 11 | 3 | 73 | 26 | 1 | 134 |
| 140 | 15 | 5 | 71 | 28 | 1 | 173 |
| 150 | 17 | 7 | 68 | 30 | 2 | 200 |
| 160 | 19 | 9 | 63 | 33 | 4 | 200 |
| 170 | 20 | 11 | 55 | 34 | 10 | 182 |

X_{MeOH}: conversion of methanol,
X_{CO}: conversion of CO,
S_{DMC}: selectivity of dimethyl carbonate formation,
S_{DMM}: selectivity of dimethoxymethane formation,
S_{DME}: selectivity of dimethyl ether formation (each based on methanol conversion), and
STY_{DMC}: space-time yield of DMC.

Following an initial operation phase of about 10 h, the STY_{DMC} determined at 150° C. decreased by less than 5 percent during the further course of the test up to an operating period of about 80 h, but showed a stable STY of 192 g/l_{cat} h for the remaining time of testing up to an operating period of 103 h.

FIG. 1 shows the results of measurements for a total operating period of about 100 h. X_{MeOH} represents the conversion of methanol, X_{CO} represents the conversion of CO, S_{DMC} represents the selectivity of dimethyl carbonate formation, STY_{DMC} represents the space-time yield of DMC in grams of DMC per liter catalyst per hour, and t represents the operating period in hours. As the apparent density of the catalyst is about 0.5, an STY of about 400 g/kg_{cat} h, based on the mass of catalyst, is obtained.

### EXAMPLE 6

Example 6 differs from Example 5 in that the DMC synthesis was performed at an overall pressure of 10 bars under otherwise identical conditions. An increase of the STY for DMC at a reaction temperature of 150° C. from 200 to 287 g of DMC I_{cat}⁻¹ h⁻¹ was found.

1. A catalyst for the synthesis of dimethyl carbonate in the gas phase, characterized in that the catalyst is a crystalline or partially amorphous aluminum silicate having the composition (based on the anhydrous form):
[Cu(I)ₐ,H_{b},Me(I)_{c},Me(II)_{d},Me(III)ₑ][(AlO₂)_{f}(SiO₂)_{g}] (1),
wherein 1≦a≦f,0≦b≦f−1, Me(I) represents a univalent cation such as Ag, Li, Na, K, Rb, Cs with 0≦c≦f−1, Me(II) represents a divalent cation such as Zn, Co, Fe, Be, Mg, Ca, Sr, Ba or Ni with 0≦d≦(f−1)/2, and Me(III) represents a trivalent cation such as Co, Fe, Cr, La with 0≦e≦(f−1)/3, the sum of the indices is a, b, c, d, e=f, the index f may assume values of from 4 to 58, and the silicon-to-aluminum ratio g/f varies from 1 to 100, and wherein Cu(I) represents a summary mean oxidation number of copper, independently of the actual oxidation numbers Cu(0), Cu(I) or Cu(II),
the content of copper in the zeolite ranging from 1 to 20 wt.-%, obtainable by
impregnation or ion exchange of a zeolite in a liquid aqueous phase with halide-free copper(II) compounds, or precipitation of Cu(II) hydroxide from an aqueous phase in the presence of said zeolite, or impregnation, ion exchange and precipitation, calcination of the produced Cu-containing zeolite in air at 300-500° C.,
and activation of the Cu-containing zeolite to give the summary mean oxidation number Cu(I) of copper in formula (I) by treatment with an inert gas at 600-900° C. or with a mixture of inert gas and water vapor at 300-900° C. until a white or virtually white solid is obtained.

2. The catalyst according to claim 1, characterized in that the halide-free copper(II) salts are selected from the group consisting of Cu(II) complexes, salts of inorganic acids, salts of organic alkanoic acids and salts of organic hydroxyalkanoic acids.

3. The catalyst according to claim 1, characterized in that the treatment is effected under inert conditions in the presence of nitrogen or noble gas for a time period of from 0.1 to 100 h.

4. The catalyst according to claim 1, characterized in that the treatment is effected under inert conditions in the presence of nitrogen or noble gas or mixtures thereof and water vapor for a time period of from 0.1 to 100 h, the water vapor being present at a volume concentration of from 0.1 to 99%.

5. The catalyst according to claim 4, characterized in that the inert treatment is effected in the presence of 5-80 vol.-% water vapor.

6. The catalyst according to claim 1, characterized in that the inert treatment is achieved in a microwave field.

7. The catalyst according to claim 1, characterized in that the inert treatment is achieved under vacuum conditions.

8. The catalyst according to claim 1, characterized in that the treatment is performed after
(i) a reduction has been effected, or (ii) further exchange of ammonium ions and protons with the cations given under Me(I), Me(II) and Me(III) has been effected.

9. The catalyst according to claim 8, characterized in that the reduction is effected using a mixture of inert gas and hydrogen, methanol, CO or a mixture thereof at 100 to 600° C., preferably in a range of from 150 to 5000° C.

10. The catalyst according to claim 1, characterized in that the hydrothermal treatment is performed at 600-800° C. using a stream of inert gas including 5-80% of added water vapor, said treatment being continued for 0.1 to 100 hours.

11. The catalyst according to claim 1, characterized in that the content of copper in the zeolite ranges from 10 to 20 wt.-%.

12. The catalyst according to claim 1, characterized in that the zeolite is selected from the group consisting of Beta, ZSM-5, mordenite, zeolite X, zeolite Y, mazzite, Omega and L.

13. The catalyst according to claim 1, characterized in that the activation with inert gas is performed at 710-850° C.

14. The catalyst according to claim 1, characterized in that the activation is performed using a mixture of inert gas and water vapor at 600-800° C.

15. The catalyst according to claim 13, characterized in that the activation is effected following reduction with a mixture of dry inert gas and hydrogen, methanol, CO or a mixture thereof at 100-600° C., preferably at 150-500° C., and subsequently with inert gas at 400-900° C.

16. The catalyst according to claim 1, characterized in that the activated catalyst is present in a mixture with an inert material having a heat conductivity of at least 10 W/m·Kelvin.

17. Use of the copper-containing zeolite catalyst according to claim 1 in a catalytic process for the synthesis of dimethyl carbonate by direct carbonylation of methanol with carbon monoxide and oxygen, the reaction of methanol with carbon monoxide and oxygen being effected at temperatures of from 120 to 220° C., advantageously from 130 to 170° C., at pressures of from 1 to 25 bars and at a gas volume load of from 500 to 5,000 h⁻¹.

18. A method for the synthesis of dimethyl carbonate by direct carbonylation of methanol with carbon monoxide and oxygen, the reaction of methanol with carbon monoxide and oxygen being effected at temperatures of from 120 to 220° C., advantageously from 130 to 170° C., at pressures of from 1 to 25 bars and at a gas volume load of from 500 to 5,000 h⁻¹ comprising performing said carbonylation with a catalyst of claim 1.
